(19) United States
(12) Patent Application Publication
(10) Pub. No.: US 2007/0049955 A1
(43) Pub. Date: Mar. 1, 2007

(54) Oral hygiene device

(75) Inventor: Scot Neal Andersen, Draper, UT (US)

Correspondence Address: RANDALL B. BATEMAN;BATEMAN IP LAW GROUP, 8 EAST BROADWAY, SUITE 550, PO BOX 1319, SALT LAKE CITY, UT, 84110, US

(73) Assignee: David R. Sharp

(21) Appl. No.: 11/217,267

(22) Filed: Sep. 1, 2005

(51) Int. Cl. A61B 17/24

(52) U.S. Cl. 606/161

(57) ABSTRACT

An oral hygiene device includes an anti-microbial compound which is applied to tissues in the mouth to freshen breath or otherwise inhibit the growth of bacteria. In one embodiment, the anti-microbial compound is combined with a tongue scraper to remove bacteria and other contaminants from the tongue prior to application of the anti-microbial compound. The anti-microbial compound typically includes a anti-microbial agent and a binding and tactifying agent, and can have flavoring, sweeteners, and abrasives added.

### BACKGROUND

1. Field of the Invention

The present invention relates to an oral hygiene device for limiting the growth of bacteria in the mouth of a user, or otherwise improve oral hygiene. More specifically, the present invention relates to an oral hygiene device which uses a anti-microbial compound for inhibiting the growth of bacteria and other microbes in the user's mouth over a prolonged period of time. In accordance with various embodiments of the invention, the anti-microbial compound can be applied from an applicator, or from a combined applicator and bacteria removal device, such as a tongue scraper.

2. State of the Art

To maintain proper hygiene, individuals engage in a number of activities to reduce bacterial count and otherwise remove contaminants from the mouth. The most common activity, of course, is using a toothbrush and toothpaste to remove plaque and food particles from one's teeth and gums to thereby lessen the risk of dental carries. The scrubbing action of the tooth brush removes most of the bacteria, etc., from the teeth and adjacent gums.

Dentists also highly recommend frequent flossing. Passing the floss between the teeth and below the gum line removes food particles caught therein. It also breaks up bacteria colonies between the teeth and gums which can cause dental carries.

While frequent brushing and flossing can significantly reduce tooth decay and periodontal disease, they do not resolve all issues of dental hygiene. For example, many people have bad breath or halitosis due to bacteria which feed on food particles in the mouth. While brushing reduces the levels of such bacteria on the teeth and associated gums, it has only a marginal effect on overall bacteria concentrations. Almost immediately after brushing has ceased, bacteria start growing again. This is readily demonstrated by slimy or filmy feeling which develops in the mouth within a relatively short time after brushing one's teeth. This is caused in large part by the large bacteria colonies present on the person's tongue and in other parts of the mouth.

To treat bad breath and otherwise reduce bacteria in the mouth, many people use mouthwash. The mouthwash also helps to wash out food particles, dead tissue cells and the like. Unfortunately, many mouthwash solutions have an unpleasant taste and provide a distinct medicine smell after use. Additionally, the fresh feeling which is provided by the mouthwash quickly dissipates and bacterial growth resumes on the tongue and other oral tissues.

Yet another problem with the common use of mouthwash is that the mouthwash contains antiseptics, such as methyl alcohol. Thus, it is recommended that the user not swallow the mouth wash. However, when thoroughly rinsing and gargling with a liquid, the natural reflex is to swallow the liquid once it is received in the back part of the mouth. For this reason, many people using mouthwash rinse only the forward portions of the mouth. While the mouthwash does kill some germs and removes some other contaminants, it generally has minimal effectiveness at removing the bacteria, dead cells, and food particles at the back of the tongue which contribute to bad breath. As soon as the mouthwash has been expelled from the mouth, these bacteria are able to work their way forward on the tongue, recontaminating the recently cleaned areas. Thus, the medicine smell of the mouth wash merely masks bad breath, and does so only temporarily.

A much less used instrument for dental hygiene is a tongue scraper. Over the years, there have been numerous different configurations of tongue scrapers which are designed to abrade the surface of the tongue and remove bacteria, dead cells, and food particles. They remove large amounts of bacteria, dead cells, and food particles along the tongue. Tongue scrapers are particularly desirable in that they remove the white film on the tongue and temporarily give a cleaner feeling to the mouth. Representative examples of tongue scrapers include U.S. Pat. Nos. 1,893,524; 2,583,750; 4,582,059; 5,569,278; 5,735,35,864; 5,766,193; 5,779,475; 5,779,654; 5,792,159; 5,810,856; 5,868,769; 5,916,228; 5,938,673; Des. 367,707; Des. 385,962; Des. 391,370; Des. 404,487; Des. 406,891; and Des. 408,534; Considerable attention is paid in these patents to the configuration of the scraper head and the manner in which it engages the tongue. Patents for tongue scrapers date back at least 130 years to U.S. Pat. No. 194,364.

While the tongue scraper removes large amounts of bacteria and food particles, the small amounts of bacteria and related food source which remain on the tongue allow bacteria colonies to quickly regrow. These bacteria contribute to the slimy feeling that can develop in the mouth and also contribute to bad breath. While attempts have been made to provide tongue scrapers which also emit mouthwash to thereby kill bacteria, the release of mouthwash provides modest relief. Despite being released from the tongue scraper, suffers the same disadvantages mentioned above for the purposes of oral hygiene.

Thus, there is a need for an improved oral hygiene device. Such a device should be easy to use and should help inhibit bacterial growth in the mouth of the user.

### SUMMARY OF THE INVENTION

It is an object of the present invention to provide an oral hygiene device that is relatively easy to use.

The above and other objects of the invention are accomplished by a oral hygiene device having an anti-microbial compound disposed thereon to kill or otherwise inhibit the growth of bacteria in the user's mouth. In accordance with one aspect of the present invention, the oral hygiene device also has a tongue scraper or other bacteria removal device disposed thereon. Preferably, the tongue scraper, etc., is used to remove the film which develops on the user's tongue. The anti-microbial compound is then used to treat the tongue or other areas of the mouth to inhibit the regrowth of bacterial colonies.

In accordance with one embodiment of the present invention, the oral hygiene device has a first end which is configured to scrape a patient's tongue and a second end which is configured to hold an anti-microbial compound for disposition on a user's tongue, or other portions of the mouth, to kill bacteria and/or inhibit the growth thereof.

In accordance with another aspect of the invention, the anti-microbial compound is disposed in the form of a generally solid compound. As the anti-microbial compound is placed against tissue in the user's mouth, the anti-microbial compound forms a layer of anti-microbial gel or film which kills bacteria or inhibits the growth thereof. In a preferred application of the invention, the anti-microbial compound is a solid which becomes hydrated or dissolved when it contacts moisture on the tongue, etc., and leaves a thin layer of anti-microbial gel on the tongue as the generally solid anti-microbial compound is moved back and forth along the tongue.

In accordance with yet another aspect of the invention, the gel is sufficiently viscous and/or sticky that it adheres to tissues in the mouth and remains on the tissue over a prolonged period of time to control the growth of bacteria and other microbes.

In accordance with another embodiment of the present invention, the tongue scraper may be omitted, and the anti-microbial compound placed on the end of an applicator for application on the tongue or adjacent tissues. As the anti-microbial compound contacts the moist tissues in the mouth, it forms a gel which is deposited on the tongue. The gel remains on the tongue for a prolonged period of time and continues to kill bacteria, thereby inhibiting the development of bad breath.

In accordance with yet another aspect of the present invention, the anti-microbial compound can include flavorings, such as spearmint, wintergreen or peppermint, which give a fresh feeling to the mouth. Such flavorings also typically have refreshing odors which will mask bad breath caused by bacterial growth.

In accordance with another aspect of the invention, the anti-microbial compound can also include sweeteners. The sweeteners help make the anti-microbial compound unobjectionable to the user, even if the gel remains on the user's tongue for a prolonged period of time.

In accordance with still another aspect of the present invention, the anti-microbial compound can include abrasives. The abrasives help to loosen dead skin cells and to break up bacterial colonies to improve penetration of the anti-microbial compound.

In accordance with still another aspect of the invention, the compound can include an anesthetic to help numb the tongue or throat of a user, such that a person suffering from a sore throat or other oral discomfort can numb the area of discomfort for a prolonged period of time.

Those of ordinary skill in the art will appreciate that there are numerous different embodiments for utilizing the various aspects of the invention and that various embodiments may resolve various problems discussed above with respect to the prior art.

### BRIEF DESCRIPTION OF THE DRAWINGS

The above and other objects, features and advantages of the invention will become apparent from a consideration of the following detailed description presented in connection with the accompanying drawings in which:

FIG. 1 shows a top view of an oral hygiene device made in accordance with the principles of the present invention;

FIG. 2 shows a side view of the oral hygiene device of FIG. 1;

FIG. 3 shows a top view of the oral hygiene device of FIGS. 1 and 2 with the anti-microbial compound removed from the device to reveal the applicator end of the device;

FIG. 4 shows a top view of an alternate embodiment of an oral hygiene device with the anti-microbial compound removed to reveal the applicator end of the device;

FIG. 4A shows a side view of the embodiment of FIG. 4 with the anti-microbial compound disposed on the applicator end;

FIG. 5 shows a side view of yet another embodiment of an oral hygiene device made in accordance with the principles of the present invention; and

FIG. 5A shows a tope view of the embodiment of the oral hygiene device shown in FIG. 5.

### DETAILED DESCRIPTION

Reference will now be made to the drawings in which the various elements of the present invention will be given numeral designations and in which the invention will be discussed so as to enable one skilled in the art to make and use the invention. It is to be understood that the following description is only exemplary of the principles of the present invention, and should not be viewed as narrowing the pending claims.

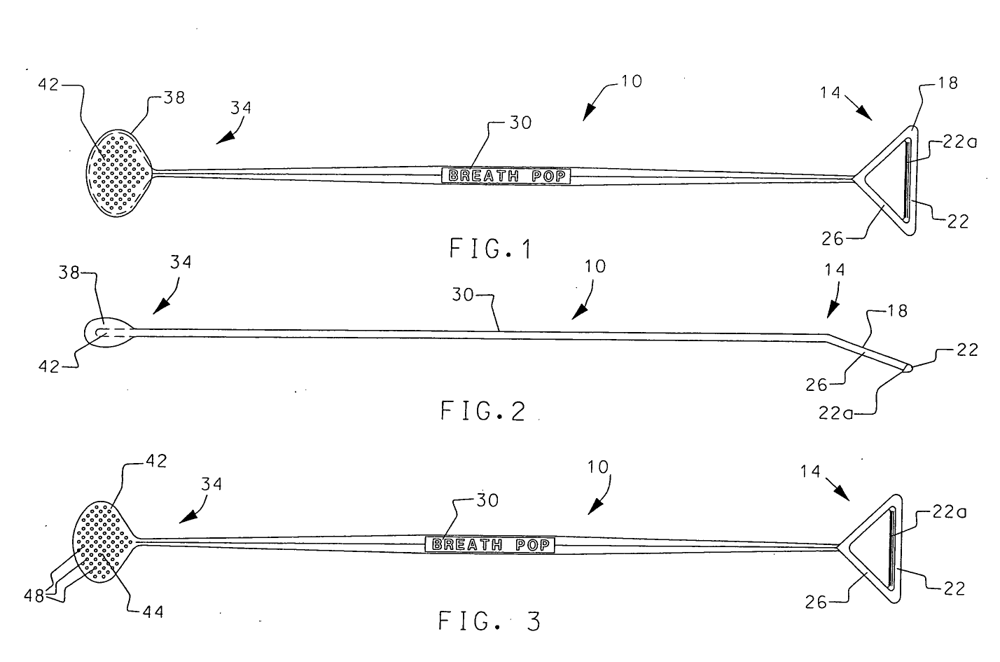

Referring to FIG. 1, there is shown a top view of an oral hygiene device, generally, indicated at 10, made in accordance with the principles of the present invention. The oral hygiene device 10 has a first end, generally indicated at 14, with a tongue scraper 18 configured thereon. The tongue scraper 18 is formed by a scraper bar 22 and a pair of angled side bars 26 which are connected to a main rail 30 of the oral hygiene device 10. The mail rail 30 is elongate and is preferably between about three and four inches long.

The scraper bar 22 and the remaining components are preferably molded out of plastic. This allows the entire oral hygiene device 10 to be disposable. It will be appreciated, however, that other materials such as metal or wood could also be used. Additionally, the oral hygiene device 10 could be cleaned and reused if desired.

The scraper bar 22 preferably is beveled on a inner edge 22a to provide a scraper edge which is dragged along the surface of the tongue of the user. As the scraper edge 22a is drawn along the tongue, the scraper edge lifts bacteria, food particles, and dead cells from the surface of the tongue. After each pass over the tongue, the scraper edge 22a can be rinsed to remove the contaminants which have been lifted from the mouth.

By repeatedly passing the scraper bar 22 over the tongue, the user is able to remove the white film and associated bacteria on the tongue. With the bacteria removed, the risk of halitosis is diminished, as the source of the foul odors is reduced. Additionally, by reducing the amount of bacteria in the mouth, the risk of dental carries and periodontal disease may be reduced.

Disposed at a second end 34 of the oral hygiene device 10 is an anti-microbial compound 38. It will be appreciated by those of skill in the art that the anti-microbial compound 38 can be either a single substance or a compound or mixture of substances. The important factor is that the anti-microbial compound inhibit microbe growth where it is applied, and that the anti-microbial compound remain on the tongue for some length of time. Those skilled in the art will be familiar with numerous anti-microbial compounds, such as polyethylene glycol, carbamide peroxide, sodium chlorite, hydrogen peroxide, sodium peroxide, sodium perborate, propyl paraben, methyl paraben, ethyl paraben, triclosan and benzalkonium chloride

In order to maintain the anti-microbial compound 38 on the oral hygiene device 10, it is preferred that the anti-microbial compound is either a solid or a semi-solid, such as a very viscous gel, hereinafter referred to as generally solid. In one embodiment of the invention, the anti-microbial compound 38 forms a solid on an applicator (shown in FIG. 1 as dashed line 42) of the oral hygiene device 10. In such a form, the anti-microbial compound 38 will not be removed from the oral hygiene device 10 to any significant degree if the user accidentally touches the second end 34 of the oral hygiene device when scraping the tongue. Additionally, the oral hygiene device can be stored in a plastic wrapper without removing most of the anti-microbial compound, and will not spill as a liquid impregnated sponge might.

Once the generally solid anti-microbial compound 38 is placed against the tongue or other tissues in the mouth, the anti-microbial compound 38 is dissolved or hydrated and forms a gel-like compound. Thus, as the second end 34 is passed over the tongue or other tissues, a layer of anti-microbial gel is deposited on the tongue. The anti-microbial gel remains on the tongue and inhibits growth of bacteria colonies. This is particularly beneficial along the back of the tongue where bacteria colonies are common and mouthwash and other hygiene products are generally ineffective for a prolonged period of time.

As the anti-microbial gel adheres to the tongue, the gel kills, or otherwise inhibits the growth of bacteria and thereby inhibits the development of odor producing compounds in the user's mouth. This is accomplished by including any of a number of known binders and tactifying agents, such as carboxypolymethylene, collidon, xantham gum, etc. While the gel will eventually dissolve and be removed from the user's tongue due to the user's saliva and swallowing, the gel will typically remain in place for at least three to fifteen minutes. This is in sharp contrast to mouthwashes which are typically present for 30 seconds or less. Thus, with a single application, a user can minimize halitosis or other problems associated with oral microorganisms for a prolonged period of time. By using the oral hygiene device 10 after each meal, a user can be substantial halitosis free during the day.

In addition to the anti-microbial compound 38 killing or inhibiting growth of bacteria, the anti-microbial compound 38 can also contain compounds which mask odors as they do develop. Such compounds are often given a strong scent such as wintergreen or peppermint. The compounds have both the effect of masking unpleasant odors and providing a refreshing taste in the user's mouth, thereby reducing the unpleasant taste which often accompanies bacterial growth.

In addition to odor masking, flavorings may be added to make the anti-microbial gel more pleasing to the user. Such flavorings could include, for example, peppermint, berry, grape, banana, wintergreen, cinnamon, spearmint, butterscotch, cherry, orange and strawberry. Other flavorings could also be used.

In addition to flavorings, sweeteners could may also be added to provide a pleasant taste. Such sweeteners could include phenylalanine, sodium saccharin, sucrose, maltitol, manitol and sorbitol. Other sweeteners could also be used.

The compound 38 may instead of or in combination with the anti-microbial include any oral topical anesthetics, such as benzocane, can be used to numb area of discomfort, such as a sore throat or a tooth ache.

The second end 34 of the oral hygiene device 10 can also have abrasives to help scour the surface of the tongue and to break up bacteria colonies thereon. The abrasives may either be positioned on the outside of the anti-microbial compound 38, or formed as part of the anti-microbial compound mixture. Abrasives such as silica powder, calcium carbonate, pumice, silicon carbide, sodium carbonate, sodium bicarbonate and gypsum powder could be used. Other abrasives could also be used.

In a presently preferred combination, the range of the various components of the anti-microbial mixture would be about:

| | |
|---|---|
| Flavors: | 0-10% |
| Sweeteners: | 0-35% |
| Anti-microbial agent: | 0.1-60% |
| Binding and tactifying agents: | 5-95% |
| Abrasives: | 0-90% |

Those skilled in the art will appreciate that amounts outside of these presently preferred ranges could be used without departing from the scope of the invention. Those skilled in the art will be able to readily adjust the various percentages of the components to develop other anti-microbial compounds which will inhibit the growth of bacteria in the user's mouth.

Turning now to FIG. 2, there is shown a side view of the oral hygiene device 10 of FIG. 1. As shown in FIG. 2, the first or scraper end 14 of the oral hygiene device 10 has a tongue scraper 18. The tongue scraper 18 are a relatively low profile to allow the scraper to reach far back into the user's mouth while minimizing the tendency to prompt a gag reflex.

FIG. 2 also shows the tongue scraper 18 being angled downwardly so that the scraper bar 22 and edge 22a are positioned below the main rail 30. The scraper bar 22 and edge 22a need not be disposed below the rail. However, this configuration is advantageous because it facilitates contact between the scraper bar 22 and the tongue, especially when the tongue scraper 18 is positioned toward the rear of the tongue. When the angle tongue scraper is not used, the user can compensate by adjusting the angle of the main rail 30 to ensure that the scraper bar 22 engages the tongue.

Disposed at the second end 34 of the oral hygiene device 10, on the opposite end of the rail 30, is the anti-microbial compound 38. The anti-microbial compound 38 is disposed on the applicator (represented by dashed line 42).

The small profile of the oral hygiene device 10 makes it inexpensive to make, easy to use and easy to discard. Unlike many of the prior art tongue scrapers, the tongue scraper can be made for pennies a piece. Thus, there is simply no need to continue to use the oral hygiene device 10 for more than one cleaning of the tongue. This, or course, eliminates the need to sanitize the tongue scraper 18 to prevent it from becoming a breeding ground for bacteria. In contrast, many of the tongue scrapers of the prior art are non-disposable and should be sanitized to prevent auto-contamination.

At the opposing second end 34 of the oral hygiene device, the anti-microbial compound 38 forms a rounded or tear drop shape on the applicator 42. As the anti-microbial compound 38 is moved across the surface of the tongue, or other tissues in the mouth, the moisture thereon will cause the anti-microbial compound to moisten and dissolve/hydrate into a gel or highly viscous material, which adheres to the surface of the tongue. The gel formed by the anti-microbial compound 38 is sufficiently viscous to remain on the tongue for a prolonged period of time. If necessary, materials such as xanthan gum or other fillers could be added to increase the viscosity of the material used to control bacterial growth and to hold it on the tongue. However, the examples set forth below effectively cling to the surface of the tongue.

Turning now to FIG. 3, there is shown a top view of the oral hygiene device 10 discussed above in FIGS. 1 and 2, with the anti-microbial compound 38 removed from the applicator 42 at the second end 34 of the device. The applicator 42 is preferably formed with a broad, rounded holding surface 44. A plurality of holes 48 are disposed in the holding surface 44 to allow anti-microbial compound on opposing sides of the holding surface to hold to each other.

The anti-microbial compound 38 can be disposed on the holding surface 44 in a variety of ways. For example, the holding surface 44 can be dipped in a gel of the anti-microbial compound 38. The anti-microbial compound 38 is then preferably dried or otherwise hardened so that the anti-microbial compound 38 does not come off in shipping, or while the user uses the tongue scraper 18 at the opposing end of the oral hygiene device. In the alternative, the anti-microbial compound can be melted, disposed on the applicator end, and then allowed to cool.

While the broadly rounded holding surface 48 shown in FIG. 3 is preferred, those skilled in the art will appreciate that numerous other shapes may be used. Additionally, while the presence of the holes 44 is preferred, they are not required. Other mechanisms for assisting in anchoring the anti-microbial compound 38 on the holding surface, such as projections or detents, may also be used.

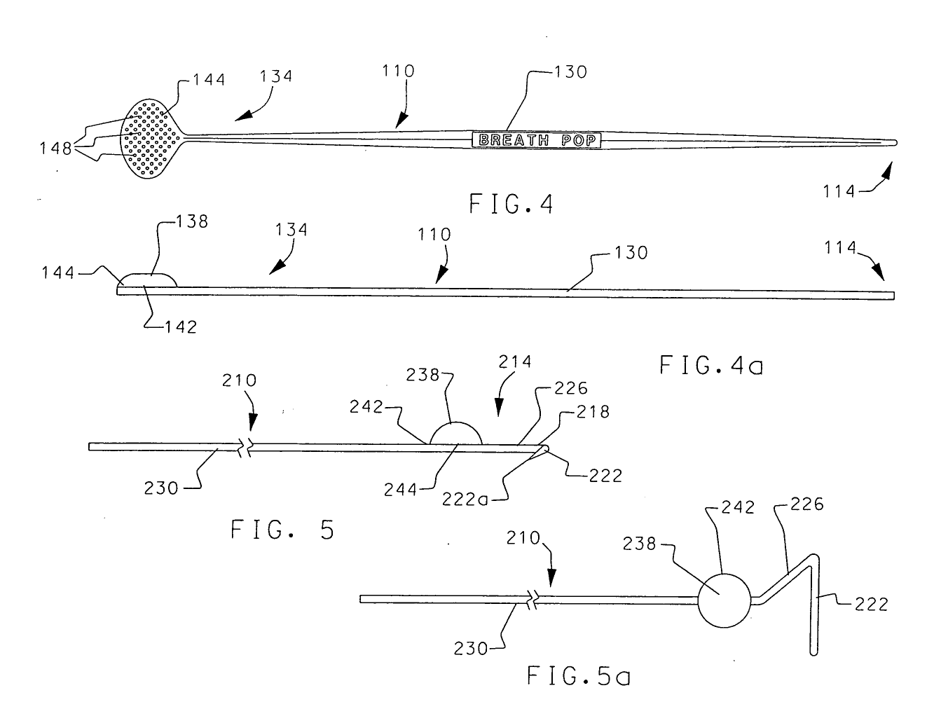

Turning now to FIG. 4, there is shown a top view of another embodiment of an oral hygiene device, generally indicated at 110, made in accordance with the principles of the present invention. The oral hygiene device 110 is similar to the oral hygiene device 10 discussed with respect to FIGS. 1 through 3, in that it has a main rail 130. At one end 134 of the rail 130 is a holding surface 144 which is configured to hold an anti-microbial compound (not show). The holding surface 44 includes a plurality of holes 148 to facilitate holding of the anti-microbial compound on the holding surface.

The opposing end 114 lacks a tongue scraper 18 (FIGS. 1 through 3). Those skilled in the art will appreciate that some people prefer using other types of tongue scrapers or simply using a tooth brush to remove the food particles and bacteria that accumulate on the tongue. The oral hygiene device 110 can then be used by grabbing the main rail 130 adjacent the first end 114 and wiping the second end 134 over the tongue and/or other tissues in the mouth. As the anti-microbial compound (not shown in FIG. 4) contacts the tongue, it leaves a thin layer of gel which both freshens the breath and inhibits the growth of bacteria.

FIG. 4A shows a side view of the oral hygiene device 110 of FIG. 4. While the anti-microbial compound 138 may be disposed on both sides of the holding surface 144 of the applicator 142, it can only be disposed on a single side if desired. Of course, the oral hygiene device could be modified to have a tongue scraper on one side of the applicator 142, and the anti-microbial compound 138 on the other side.

Turning now to FIG. 5, there is shown a side view of yet another embodiment of an oral hygiene device, generally indicated at 210, in accordance with the principles of the present invention. The oral hygiene device 210 has a first end 214 which forms a scraper bar 218. The tongue scraper 218 includes a scraper bar 222 which is used to scrape food particles, bacteria and dried saliva from the tongue. If desired, the scraper bar 222 can have and edge 222a which is tapered to cut through the film on the tongue and help lift it away from the tongue as the scraper is moved toward the front of the user's mouth.

The scraper bar 222 is attached by an arm 226 to a main rail 230. The main rail 230 is preferably at lest two inches long, and more preferably between three and four inches long to enable to tongue scraper 218 to be extended to a position adjacent the back of the tongue.

Disposed on the main rail 230 slightly distally from the tongue scraper 218 is an anti-microbial compound 238. The anti-microbial compound 238 is typically supported by an applicator 242 which forms a holding surface 244. The holding surface 244 can be generally planar, or can have a plurality of holes, projections, detents or other mechanical or chemical structures for helping to hold the anti-microbial compound 238 to the holding surface until it is deposited in the user's mouth.

In use, the tongue scraper 218 is pulled repeatedly across the tongue until the desired film, etc., has been removed. The oral hygiene device 210 is then turned over so that the anti-microbial compound 238 can be applied to the tongue. If desired, the anti-microbial compound can also be applied to the teeth, gums and other tissues in the mouth.

FIG. 5A shows a top view of the oral hygiene device of FIG. 5. The tongue scraper 218 is configured somewhat differently than the tongue scraper 218 shown in FIGS. 1 through 3. Such a configuration allows the tongue scraper 218 to more easily reach some locations on the tongue and other mouth tissues. It is important to note that the configuration of other tongue scrapers can also be used in accordance with the principles of the present invention.

As demonstrated in FIGS. 5 and 5A, it is not critical that the anti-microbial compound 238 be at an opposing end of the oral hygiene device 210 from the tongue scraper 218. While such a configuration minimizes the thickness of the end with the tongue scraper, some people will not like handling the first end 14 (FIG. 1) of the oral hygiene device—which can been coated with the removed contamination—while applying the anti-microbial compound.

In the embodiment shown in FIG. 5, the user's hand need not come into contact with the portion having the tongue scraper 218. Once finished with the tongue scraper 218, the user simply rotates the main rail 230 180 degrees about its longitudinal axis, and rubs the anti-microbial compound 238 on the tongue.

A variety of different compounds can be used as the anti-microbial compound 38 or 238. The following are examples of antimicrobial agents which can be used on the applicator 42 or 242 of the oral hygiene device 10. In light of the present disclosures, those skilled in the art will appreciate that numerous other compounds and mixtures could be used on the applicator 42 or 242 in accordance with the principles of the present invention. For example anti-microbial agents such as carbamide peroxide, sodium chlorite, hydrogen peroxide, sodium peroxide, sodium perborate, propyl paraben, methyl paraben, ethyl paraben, triclosan and benzalkonium chloride can be included. Other anti-microbial agents may also be used. Those skilled in the art will appreciate that such anti-microbial agents usually have anti-bacterial properties, and also reduce the slimy feeling which bacterial growth can cause in the mouth.

Thus, there is disclosed an improved oral hygiene device. Those skilled in the art will appreciate that numerous modifications can be made to the embodiments shown herein. For example, in accordance with the principles of the present invention, the anti-microbial compound could be used with other tongue scrapers or tooth brushes to inhibit bacterial growth in the mouth.

Those skilled in the art with also appreciate that the embodiments discussed above are only meant to teach one of ordinary skill in the art how to use the invention and not to limit the claims. The appended claims are intended to cover modifications.

What we claim is:

1. An oral hygiene device comprising:
an applicator for applying an anti-microbial compound to the mouth of a user; and
an anti-microbial compound in the form of a solid or semi-solid, such as a gel, the anti-microbial compound comprising at least one compound which inhibits bacterial growth.

2. The oral hygiene device according to claim 1, wherein the anti-microbial compound is a gel when in contact with moisture in the mouth of a user.

3. The oral hygiene device according to claim 1, wherein the anti-microbial compound comprises a anti-microbial agent.

4. The oral hygiene device according to claim 1, wherein the anti-microbial compound comprises a binding and tactifying agent.

5. The oral hygiene device according to claim 1, wherein the anti-microbial compound comprises flavoring.

6. The oral hygiene device according to claim 1, wherein the anti-microbial compound comprises sweeteners.

7. The oral hygiene device according to claim 1, wherein the anti-microbial compound comprises abrasives.

8. The oral hygiene device according to claim 1, wherein the anti-microbial compound comprises an anesthetic.

9. The oral hygiene device according to claim 1, further comprising a tongue scraper.

10. The oral hygiene device according to claim 9, wherein the tongue scraper is disposed at a first end of the oral hygiene device and wherein the anti-microbial compound is disposed at a second end of the oral hygiene device.

11. The oral hygiene device according to claim 1, wherein the applicator comprises a holding surface.

12. The oral hygiene device according to claim 11, wherein the holding surface has a plurality of holes formed therein.

13. An oral hygiene device comprising:
a first end having a tongue scraper formed thereat;
a second end forming an applicator for holding and applying an anti-microbial compound; and
a generally solid anti-microbial compound disposed on the second end.

14. The oral hygiene device according to claim 13, wherein the generally solid anti-microbial compound dissolves to form a gel when coming into contact with saliva.

15. The oral hygiene device according to claim 13, wherein the anti-microbial compound is a dried gel.

16. The oral hygiene device according to claim 13, wherein the anti-microbial compound comprises an anti-microbial agent, and a binding and tactifying agent.

17. The oral hygiene device according to claim 13, wherein the anti-microbial compound further comprises flavoring.

18. The oral hygiene device according to claim 17, wherein the anti-microbial compound further comprises sweeteners.

19. The oral hygiene device according to claim 17, wherein the anti-microbial compound further comprises abrasives.

20. The oral hygiene device according to claim 13, further comprising an anesthetic.

21. An oral hygiene device comprising;
a tongue scraper; and
an anti-microbial compound for application to the user's tongue, the anti-microbial compound being a solid or semi-solid.

22. The oral hygiene device according to claim 21, wherein the anti-microbial compound is a gel when exposed to moisture.

23. The oral hygiene device according to claim 21, wherein the anti-microbial compound comprises a anti-microbial agent and a binding and tactifying agent.

24. A method for inhibiting the growth of bacteria in the mouth, the method comprising;
coating the tongue with an anti-microbial compound in the form of the gel, and allowing the gel to remain on the tongue for a prolonged period of time.

25. The method for inhibiting the growth of bacteria according to claim 24, wherein the method comprises passing a tongue scraper over the tongue prior to application of the anti-microbial compound.

26. The method for inhibiting growth of bacteria according to claim 24, wherein the method comprises selecting an anti-microbial compound comprising at least one anti-microbial agent and at least one binding and tactifying agent.

27. The method for inhibiting growth of bacteria according to claim 24, wherein the method comprises selecting an anti-microbial compound having an anesthetic

28. The method for inhibiting growth of bacteria according to claim 27, wherein the method comprises selecting an anti-microbial compound having abrasives.
